(19) United States
(12) Patent Application Publication
(10) Pub. No.: US 2018/0296719 A1
(43) Pub. Date: Oct. 18, 2018

(54) AIR FRESHENING DEVICE UTILIZING A SOLID-FORM COMPOSITION AND A METHOD OF FRESHENING THE AIR

(71) Applicant: The Procter & Gamble Company, Cincinnati, OH (US)

(72) Inventors: Richard Lee LANE, Cincinnati, OH (US); William Michael CANNON, West Harrison, OH (US); Gayle Marie FRANKENBACH, Cincinnati, OH (US)

(21) Appl. No.: 15/939,348

(22) Filed: Mar. 29, 2018

Related U.S. Application Data
(60) Provisional application No. 62/486,498, filed on Apr. 18, 2017.

(51) Int. Cl. A61L 9/12; A61L 9/04

(52) U.S. Cl. CPC A61L 9/122; A61L 2209/133; A61L 9/042; A61L 9/044

(57) ABSTRACT

An air freshening device is provided. The device includes a housing and a fan connected with the housing. The device may include a pouch disposed in air-flow communication with the fan. The pouch may be disposed downstream from the fan. The pouch includes a solid-form composition enclosed by an air-permeable substrate. The device may include a screen in air flow communication with the fan. The screen may include a plurality of openings and a semi-solid or solid form composition disposed on at least a portion of the screen. The solid-form composition includes a perfume mixture and a malodor reduction material.

### FIELD

The present disclosure is generally directed an air freshening device and method of freshening the air, and, more particularly, is directed to an air freshening devices that utilizes a solid-form composition and fan and a method of freshening the air.

### BACKGROUND

Many air freshening devices exist. Some air freshening devices deliver freshness to the air by melting wax articles that comprise air freshening compositions. However, such devices can have drawbacks. For example, melted wax can spill on the device or nearby surfaces. Moreover, it may be difficult or messy to replace the wax after it has been melted. As a result, it would be beneficial to provide an air freshening device that utilizes a solid-form composition while overcoming the potential drawbacks associated therewith.

SUMMARY

### “Combinations:”

A. An air freshening device comprising:

a housing;

a fan connected with the housing; and

a pouch disposed in air-flow communication with the fan, wherein the pouch is disposed downstream from the fan, wherein the pouch comprises a solid-form composition enclosed by an air-permeable substrate, the solid form composition comprising a perfume mixture and a malodor reduction material.
B. The air freshening device of Paragraph A, wherein the solid-form composition comprises a carrier.
C. The air freshening device of Paragraph A or B, wherein the malodor reduction material comprises a material selected from the group consisting of Claim 3.
D. The air freshening device of any of Paragraphs A through C, wherein the malodor reduction material comprises a material selected from the group consisting of: odor neutralizing materials, odor blocking materials, odor masking materials, sensory modifying materials, and combinations thereof.
E. The air freshening device of any of Paragraphs A through D further comprising a cover, wherein the screen encloses the pouch in the housing.
F. The air freshening device of any of Paragraphs A through E, wherein the solid-form composition comprises about 0.1% to about 20%, by weight of the solid form composition, of perfume.
G. The air freshening device of any of Paragraphs A through F, wherein the solid-form composition comprises about 0.005% to about 10%, by weight of the solid-form composition, of malodor reduction material.
H. The air freshening device of any of Paragraphs A through G, wherein the air-permeable substrate comprises a fibrous web having an apparent opening size according to ASTM D4751-12 smaller than about 1000 μm.
I. An air freshening device comprising:

a housing;

a fan connected with the housing; and

a screen in air flow communication with the fan, wherein the screen comprises a plurality of openings and a semi-solid or solid form composition disposed on at least a portion of the screen, wherein the solid-form composition comprises a perfume mixture and a malodor reduction material.
J. The air freshening device of Paragraph I, wherein the solid-form composition completely surrounds the screen.
K. The air freshening device of any of Paragraphs I through J, wherein the solid-form composition comprises a carrier.
L. The air freshening device of any of Paragraphs I through K, wherein the solid-form composition forms a plurality of openings around the plurality of openings of the screen.
M. The air freshening device of any of Paragraphs I through L, wherein the malodor reduction material comprises a material selected from the group consisting of Claim 13.
N. The air freshening device of any of Paragraphs I through M, wherein the malodor reduction material comprises a material selected from the group consisting of: odor neutralizing materials, odor blocking materials, odor masking materials, sensory modifying materials, and combinations thereof.
O. The air freshening device of any of Paragraphs I through N further comprising a cover, wherein the cover encloses the screen in the housing.
P. The air freshening device of any of Paragraphs I through O, wherein the solid-form composition comprises about 0.1% to about 20%, by weight of the solid form composition, of perfume.
Q. The air freshening device of any of Paragraphs I through P, wherein the solid-form composition comprises about 0.005% to about 10%, by weight of the solid-form composition, of malodor reduction material.
R. A method of freshening the air with the air freshening device of any of Paragraphs A through H, wherein the method includes the step of:

providing the air freshening device;

inserting a pouch into the housing; and

turning on power to fan.
S. A method of freshening the air with the air freshening device of any of Paragraphs I through Q, wherein the method includes the step of:

providing the air freshening device;

inserting a screen into the housing; and

turning on power to fan.
T. The method of Paragraph R further comprising the step of replacing the pouch with a new pouch.
U. The method of Paragraph S further comprising the step of replacing the screen with a new screen.
V. A method of freshening the air, the method comprising the steps of:

providing a pouch, wherein the pouch comprises a solid-form composition enclosed by an air-permeable substrate, the solid form composition comprising a perfume mixture and a malodor reduction material;

placing the pouch in a space; and

allowing the perfume and malodor reduction materials to freshen the air.
W. The method of Paragraph V, wherein the solid-form composition is in the form of particles.
X. The method of any of Paragraphs V through W, wherein the malodor reduction material comprises a material selected from the group consisting of Claim 24.
Y. The method of any of Paragraphs V through X, wherein the malodor reduction material comprises a material selected from the group consisting of: odor neutralizing materials, odor blocking materials, odor masking materials, sensory modifying materials, and combinations thereof.
Z. The method of any of Paragraphs V through Y, wherein the air-permeable substrate comprises a fibrous web having an apparent opening size according to ASTM D4751-12 smaller than about 1000 μm.
AA. The air freshening device of any of Paragraphs A through Q, wherein the malodor reduction materials may be selected from the group consisting of: 1,1,2,3,3-pentamethyl-1,2,3,5,6,7-hexahydro-4H-inden-4-one, (E)-3,7-dimethylocta-2,6-dien-1-yl palmitate, (3R,3aR,6S,7S,8aS)-6-methoxy-3,6,8,8-tetramethyloctahydro-1H-3a,7-methanoazulene, Flor acetate, 1-((2-(tert-butyl)cyclohexyl)oxy)butan-2-ol, 3-Methyl-5-phenylpentanol, 2-(8-isopropyl-6-methylbicyclo[2.2.2]oct-5-en-2-yl)-1,3-dioxolane, 2,2,6,6,7,8,8-heptamethyldecahydro-2H-indeno[4,5-b]furan, 3,3-dimethyl-2,3-dihydro-1H-Indene-5-yl propanal, 1,1-dimethyl-2,3-dihydro-1H-inden-5-yl)propanal, 2,4-dimethyl-4,4a,5,9b-tetrahydroindeno[1,2-d][1,3]dioxine, (E)-oxacyclohexadec-13-en-2-one, (R,Z)-1-(2,6,6-trimethylcyclohex-2-en-1-yl)pent-1-en-3-one, 3-(7,7-dimethyl-4-bicyclo[3.1.1]hept-3-enyl)-2,2-dimethyl propanal, (Z)-cyclooct-4-en-1-yl methyl carbonate, octahydro-2H-chromen-2-one, 7-methyloctyl acetate, ethyl (2,3,6-trimethylcyclohexyl) carbonate, ethyl dodecanoate, (1R,4R,6R,10S)-4,12,12-trimethyl-9-methylene-5-oxatricyclo[8.2.0.04,6]dodecane, 4,8-dimethyl-1-(methylethyl)-7-oxybiciclo [4.3.0]nonane, 3a,4,5,6,7,7a-hexahydro-1H-4,7-methanoinden-6-yl propionate, 1-(5,5-dimethylcyclohex-1-en-1-yl)pent-4-en-1-one, 5-Cyclohexadecen-1-one, (E)-dec-4-enal, (1-methyl-2-((1,2,2-trimethylbicyclo[3.1.0]hexan-3-yl)methyl)cyclopropyl)methanol, 4-(tert-butyl)cyclohexan-1-ol, methyl palmitate, 2-isopropyl-5-methylphenol, 2,6-di-tert-butyl-4-methylphenol, and combinations thereof.
BB. The method according to any of paragraphs R through Z, wherein the malodor reduction materials are selected from the group consisting of: 1,1,2,3,3-pentamethyl-1,2,3,5,6,7-hexahydro-4H-inden-4-one, (E)-3,7-dimethylocta-2,6-dien-1-yl palmitate, (3R,3aR,6S,7S,8aS)-6-methoxy-3,6,8,8-tetramethyloctahydro-1H-3a,7-methanoazulene, Flor acetate, 1-((2-(tert-butyl)cyclohexyl)oxy)butan-2-ol, 3-Methyl-5-phenylpentanol, 2-(8-isopropyl-6-methylbicyclo[2.2.2]oct-5-en-2-yl)-1,3-dioxolane, 2,2,6,6,7,8,8-heptamethyldecahydro-2H-indeno[4,5-b]furan, 3,3-dimethyl-2,3-dihydro-1H-Indene-5-yl propanal, 1,1-dimethyl-2,3-dihydro-1H-inden-5-yl)propanal, 2,4-dimethyl-4,4a,5,9b-tetrahydroindeno[1,2-d][1,3]dioxine, (E)-oxacyclohexadec-13-en-2-one, (R,Z)-1-(2,6,6-trimethylcyclohex-2-en-1-yl)pent-1-en-3-one, 3-(7,7-dimethyl-4-bicyclo[3.1.1]hept-3-enyl)-2,2-dimethyl propanal, (Z)-cyclooct-4-en-1-yl methyl carbonate, octahydro-2H-chromen-2-one, 7-methyloctyl acetate, ethyl (2,3,6-trimethylcyclohexyl) carbonate, ethyl dodecanoate, (1R,4R,6R,10S)-4,12,12-trimethyl-9-methylene-5-oxatricyclo[8.2.0.04,6]dodecane, 4,8-dimethyl-1-(methylethyl)-7-oxybiciclo [4.3.0]nonane, 3a,4,5,6,7,7a-hexahydro-1H-4,7-methanoinden-6-yl propionate, 1-(5,5-dimethylcyclohex-1-en-1-yl)pent-4-en-1-one, 5-Cyclohexadecen-1-one, (E)-dec-4-enal, (1-methyl-2-((1,2,2-trimethylbicyclo[3.1.0]hexan-3-yl)methyl)cyclopropyl)methanol, 4-(tert-butyl)cyclohexan-1-ol, methyl palmitate, 2-isopropyl-5-methylphenol, 2,6-di-tert-butyl-4-methylphenol, and combinations thereof.

### BRIEF DESCRIPTION OF THE DRAWINGS

FIG. 1 is a perspective view of an air freshening device that utilizes a solid-form article.

FIG. 2 is a sectional view of FIG. 1 taken along lines 2-2

FIG. 3 is a perspective view of FIG. 2.

FIG. 4 is a top, plan view of a sleeve comprising a pouch of particles.

FIG. 5 is a pouch comprising a plurality of solid-form composition particles.

FIG. 6 is a sectional view of FIG. 5 taken along lines 6-6.

FIG. 7 is perspective view of a photograph of an air freshening device with a solid-form article comprising a screen having a solid-form composition surrounding the screen.

FIG. 8 is a perspective view of a photograph of the screen having the solid-form composition of FIG. 7.

FIG. 9 is a perspective view of a photograph of the air freshening device of FIG. 7 with the screen removed.

FIG. 10 is a perspective view of a photograph of a screen for an air freshening device.

FIG. 11 is a perspective view of a photograph of a container for an air freshening device.

FIG. 12 is a perspective view of a photograph of a container for an air freshening device.

### DETAILED DESCRIPTION

### Air Freshening Device

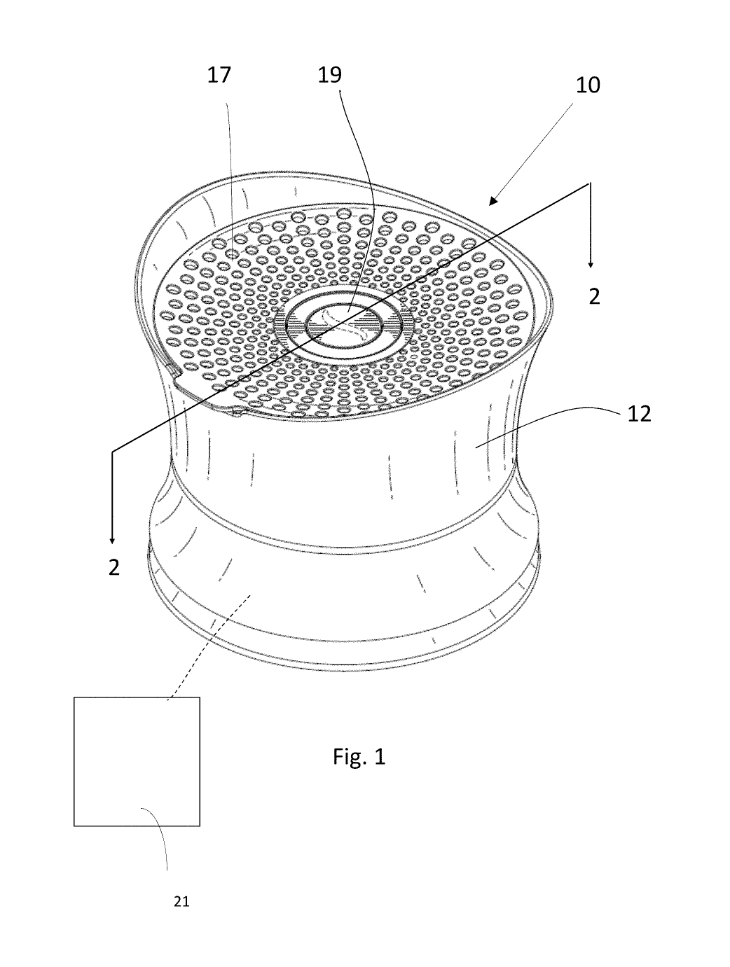

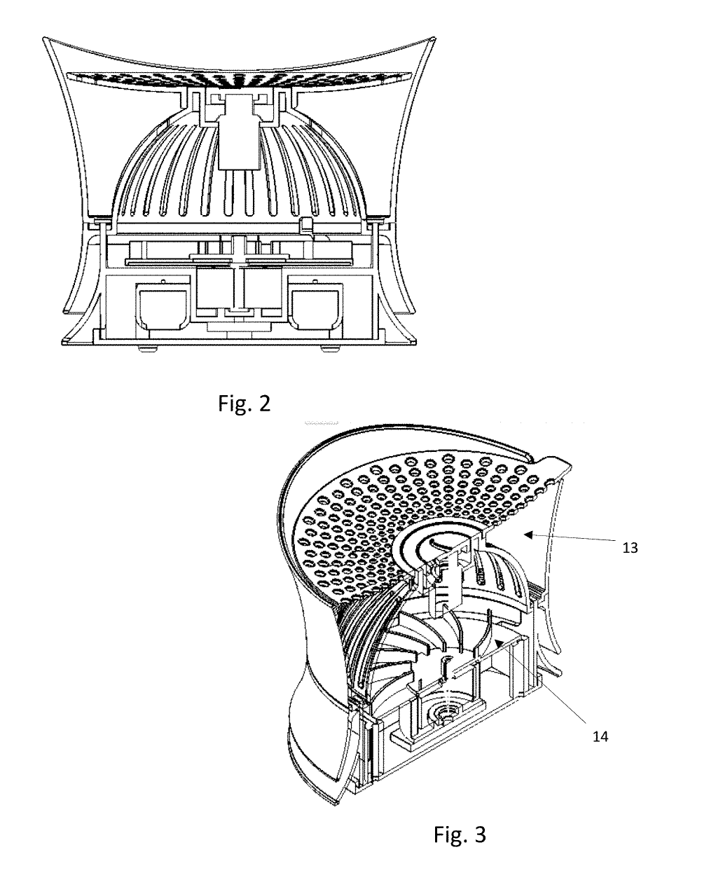

With reference to FIGS. 1-3, an air freshening device 10 may comprise a housing 12 having a chamber 13 that is configured to receive a solid-form composition. The air freshening device 10 also includes a fan 14 disposed upstream of the chamber 13 of the housing 12. The fan 14 may be electrically connectable to a power source 21. The power source may comprise batteries or an electrical wall outlet. The power source 21 may be internal or external to the air freshening device 10. The air freshening device may also comprise a cover 17 that encloses the solid-form form composition within the chamber 13. It has been found that the perfume and malodor reduction materials of the solid-form composition are able to freshen the air with the introduction of a fan to disperse the perfume and malodor reduction materials into the air.

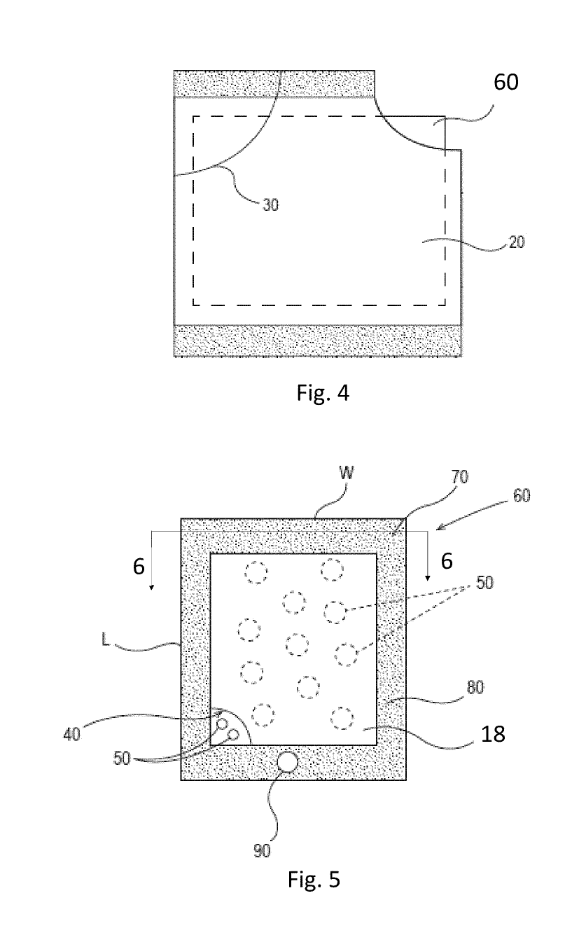

With reference to FIGS. 4 and 5, and as will be discussed in more detail below, the air freshening device 10 may comprise a pouch 60 comprising a solid-form composition in the form of particles 50 that are contained with an air-permeable substrate 18. The pouch 60 may be configured such that air flow from the fan is able to pass through the air-permeable substrate and the particles to deliver perfume and/or malodor reduction materials into the air.

The solid-form composition may be disposed on a rigid or semi-rigid screen. The screen comprising the solid-form composition may be configured such that air flow from the fan is able to pass through the plurality of openings in the screen and/or solid-form composition to deliver perfume and/or malodor reduction materials into the air.

### Solid-Form Composition

The solid-form composition may comprise a carrier and perfume. The solid-form composition may comprise a carrier, perfume, and a malodor control composition.

The carrier may be selected from the group consisting of water soluble organic alkali metal salt, water soluble inorganic alkaline earth metal salt, water soluble organic alkaline earth metal salt, water soluble carbohydrate, water soluble silicate, water soluble urea, starch, clay, water insoluble silicate, citric acid carboxymethyl cellulose, fatty acid, fatty alcohol, glyceryl diester of hydrogenated tallow, glycerol, polyethylene glycol, and combinations thereof. Alkali metal salts can be, for example, selected from the group consisting of salts of lithium, salts of sodium, and salts of potassium, and any combination thereof. Useful alkali metal salts can be, for example, selected from the group consisting of alkali metal fluorides, alkali metal chlorides, alkali metal bromides, alkali metal iodides, alkali metal sulfates, alkali metal bisulfates, alkali metal phosphates, alkali metal monohydrogen phosphates, alkali metal dihydrogen phosphates, alkali metal carbonates, alkali metal monohydrogen carbonates, alkali metal acetates, alkali metal citrates, alkali metal lactates, alkali metal pyruvates, alkali metal silicates, alkali metal ascorbates, and combinations thereof. Alkali metal salts can be selected from the group consisting of, sodium fluoride, sodium chloride, sodium bromide, sodium iodide, sodium sulfate, sodium bisulfate, sodium phosphate, sodium monohydrogen phosphate, sodium dihydrogen phosphate, sodium carbonate, sodium hydrogen carbonate, sodium acetate, sodium citrate, sodium lactate, sodium tartrate, sodium silicate, sodium ascorbate, potassium fluoride, potassium chloride, potassium bromide, potassium iodide, potassium sulfate, potassium bisulfate, potassium phosphate, potassium monohydrogen phosphate, potassium dihydrogen phosphate, potassium carbonate, potassium monohydrogen carbonate, potassium acetate, potassium citrate, potassium lactate, potassium tartrate, potassium silicate, potassium, ascorbate, and combinations thereof. Alkaline earth metal salts can be selected from the group consisting of salts of magnesium, salts of calcium, and the like, and combinations thereof. Alkaline earth metal salts can be selected from the group consisting of alkaline metal fluorides, alkaline metal chlorides, alkaline metal bromides, alkaline metal iodides, alkaline metal sulfates, alkaline metal bisulfates, alkaline metal phosphates, alkaline metal monohydrogen phosphates, alkaline metal dihydrogen phosphates, alkaline metal carbonates, alkaline metal monohydrogen carbonates, alkaline metal acetates, alkaline metal citrates, alkaline metal lactates, alkaline metal pyruvates, alkaline metal silicates, alkaline metal ascorbates, and combinations thereof. Alkaline earth metal salts can be selected from the group consisting of magnesium fluoride, magnesium chloride, magnesium bromide, magnesium iodide, magnesium sulfate, magnesium phosphate, magnesium monohydrogen phosphate, magnesium dihydrogen phosphate, magnesium carbonate, magnesium monohydrogen carbonate, magnesium acetate, magnesium citrate, magnesium lactate, magnesium tartrate, magnesium silicate, magnesium ascorbate, calcium fluoride, calcium chloride, calcium bromide, calcium iodide, calcium sulfate, calcium phosphate, calcium monohydrogen phosphate, calcium dihydrogen phosphate, calcium carbonate, calcium monohydrogen carbonate, calcium acetate, calcium citrate, calcium lactate, calcium tartrate, calcium silicate, calcium ascorbate, and combinations thereof. Inorganic salts, such as inorganic alkali metal salts and inorganic alkaline earth metal salts, do not contain carbon. Organic salts, such as organic alkali metal salts and organic alkaline earth metal salts, contain carbon. The organic salt can be an alkali metal salt or an alkaline earth metal salt of sorbic acid (i.e., asorbate). Sorbates can be selected from the group consisting of sodium sorbate, potassium sorbate, magnesium sorbate, calcium sorbate, and combinations thereof.

The carrier can be or comprise a material selected from the group consisting of a water-soluble inorganic alkali metal salt, a water-soluble organic alkali metal salt, a water-soluble inorganic alkaline earth metal salt, a water-soluble organic alkaline earth metal salt, a water-soluble carbohydrate, a water-soluble silicate, a water-soluble urea, and combinations thereof. The carrier or water soluble-soluble carrier can be selected from the group consisting of sodium chloride, potassium chloride, calcium chloride, magnesium chloride, sodium sulfate, potassium sulfate, magnesium sulfate, sodium carbonate, potassium carbonate, sodium hydrogen carbonate, potassium hydrogen carbonate, sodium acetate, potassium acetate, sodium citrate, potassium citrate, sodium tartrate, potassium tartrate, potassium sodium tartrate, calcium lactate, water glass, sodium silicate, potassium silicate, dextrose, fructose, galactose, isoglucose, glucose, sucrose, raffinose, isomalt, xylitol, candy sugar, coarse sugar, and combinations thereof. In one embodiment, the carrier or water-soluble carrier can be sodium chloride. In one embodiment, the carrier or water-soluble carrier can be table salt.

The carrier can be or comprise a material selected from the group consisting of sodium bicarbonate, sodium sulfate, sodium carbonate, sodium formate, calcium formate, sodium chloride, sucrose, maltodextrin, corn syrup solids, corn starch, wheat starch, rice starch, potato starch, tapioca starch, clay, silicate, citric acid carboxymethyl cellulose, fatty acid, fatty alcohol, glyceryl diester of hydrogenated tallow, glycerol, and combinations thereof.

The carrier can be selected from the group consisting of water soluble organic alkali metal salt, water soluble inorganic alkaline earth metal salt, water soluble organic alkaline earth metal salt, water soluble carbohydrate, water soluble silicate, water soluble urea, starch, clay, water insoluble silicate, citric acid, carboxymethyl cellulose, fatty acid, fatty alcohol, glyceryl diester of hydrogenated tallow, glycerol, polyvinyl alcohol, polyethylene glycol, and combinations thereof.

The carrier can be selected from the group consisting of polyvinyl alcohol, modified polyvinyl alcohol, polyvinyl pyrrolidone, polyvinyl alcohol/polyvinyl pyrrolidone, polyvinyl alcohol/polyvinyl amine, partially hydrolyzed polyvinyl acetate, polyalkylene oxide, polyethylene glycol, acrylamide, acrylic acid, cellulose, alkyl cellulosics, methyl cellulose, ethyl cellulose, propyl cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides, starch, modified starch, gelatin, alginates, xyloglucans, hemicellulosic polysaccharides, xylan, glucuronoxylan, arabinoxylan, mannan, glucomannan, galactoglucomannan, natural gums, pectin, xanthan, carrageenan, locus bean, arabic, tragacanth, polyacrylates, sulfonated polyacrylates, water-soluble acrylate copolymers, alkylhydroxy cellulosics, methylcellulose, carboxymethylcellulose sodium, modified carboxy-methylcellulose, dextrin, ethylcellulose, propylcellulose, hydroxyethyl cellulose, hydroxypropyl methylcellulose, maltodextrin, polymethacrylates, polyvinyl alcohol copolymers, hydroxypropyl methyl cellulose, and mixtures thereof.

The carrier can be polyethylene glycol having a weight average molecular weight between from about 2000 to about 13000.

The carrier can be formed into water soluble particles that carry the perfume. The particles can be formed by a rotoforming process. The particles can be formed on a SANDVIK ROTOFORM 3000 having a 750 mm wide 10 m long belt. The rotoforming cylinder can have 2 mm diameter apertures set at 10 mm pitch in the cross machine direction and 9.35 mm pitch in the machine direction. The rotoforming cylinder be set at about 3 mm above the belt. The belt speed and rotational speed of the rotoforming cylinder can be set at 10 m/min. A melt of the precursor material for the particles can be pumped to the rotoforming cylinder at a rate of about 3.1 kg/min from a mixer. A plate and frame heat exchanger can be set to control the temperature to be about 50 degrees Celsius.

The solid-form composition can comprise from about 40% by weight to about 99%, by weight of the particles, of the carrier. The carrier can be polyethylene glycol.

The solid-form composition can comprise more than about 40% by weight polyethylene glycol having a weight average molecular weight from about 2000 to about 13000. Polyethylene glycol (PEG) has a relatively low cost, may be formed into many different shapes and sizes, minimizes unencapsulated perfume diffusion, and dissolves well in water. PEG comes in various weight average molecular weights. A suitable weight average molecular weight range of PEG includes from about 2,000 to about 13,000, from about 4,000 to about 12,000, alternatively from about 5,000 to about 11,000, alternatively from about 6,000 to about 10,000, alternatively from about 7,000 to about 9,000, alternatively combinations thereof. PEG is available from BASF, for example PLURIOL E 8000.

The solid-form composition can comprise more than about 40% by weight of the particles of PEG. The solid-form composition can comprise more than about 50% by weight of the solid-form composition, or particles if employed, of PEG. The solid-form composition can comprise more than about 60% by weight of the solid-form composition, or particles if employed, of PEG. The solid-form composition may comprise from about 65% to about 99% by weight of the solid-form composition, or particles if employed of PEG. The solid-form composition may comprise from about 40% to about 99% by weight of the solid-form composition, or particles if employed, of PEG.

Alternatively, the solid-form composition can comprise from about 40% to about 90%, alternatively from about 45% to about 75%, alternatively from about 50% to about 70%, alternatively combinations thereof and any whole percentages or ranges of whole percentages within any of the aforementioned ranges, of PEG by weight of the solid-form composition, or particles if employed.

Depending on the application, the solid-form composition can comprise from about 0.5% to about 5% by weight of the solid-form composition of a balancing agent selected from the group consisting of glycerin, polypropylene glycol, isopropyl myristate, dipropylene glycol, 1,2-propanediol, and PEG having a weight average molecular weight less than 2,000, and mixtures thereof.

The particles may comprise dye. The particles may comprise less than about 0.1%, alternatively about 0.001% to about 0.1%, alternatively about 0.01% to about 0.02%, alternatively combinations thereof and any hundredths of percent or ranges of hundredths of percent within any of the aforementioned ranges, of dye by weight of the particles. Examples of suitable dyes include, but are not limited to, LIQUITINT PINK AM, AQUA AS CYAN 15, and VIOLET FL, available from Milliken Chemical.

The solid-form composition can comprise an antioxidant. The antioxidant can help to promote stability of the color and or odor of the solid-form composition, or particles if employed, over time between production and use. The solid-form composition can comprise between about 0.01% to about 1% by weight of the solid-form composition, or particles if employed, antioxidant. The solid-form composition can comprise between about 0.001% to about 2% by weight of the solid-form composition, or particles if employed, antioxidant. The solid-form composition can comprise between about 0.01% to about 0.1% by weight of the solid-form composition, or particles if employed, antioxidant. The antioxidant can be butylated hydroxytoluene.

The perfume can comprise one or both of unencapsulated perfume and encapsulated perfume. The perfume can be perfume provided by a perfume delivery technology, or a perfume provided in some other manner. Perfumes are generally described in U.S. Pat. No. 7,186,680 at column 10, line 56, to column 25, line 22. The perfume can be carried by a perfume carrier material. Examples of perfume carrier materials are described in U.S. Pat. No. 7,186,680, column 25, line 23, to column 31, line 7. Specific examples of perfume carrier materials may include cyclodextrin and zeolites.

The perfume can comprise a perfume raw material having a saturation vapor pressure greater than about 0.01 torr. Such a vapor pressure can be practical for having the perfume be sufficiently volatile to reach the consumers nose when the particles are in use. The composition can comprise a perfume raw material having a logP greater than about 3. Such a logP for the perfume can be practical for having acceptable deposition onto a laundry article, article of clothing. The perfume can comprise a perfume raw material having a saturation vapor pressure greater than about 0.01 torr and a logP greater than about 3. Such a perfume can be practical for providing sufficient volatility for the perfume to reach the consumers nose and sufficient deposition on to a laundry article, article of clothing, textile, or the like.

The saturation Vapor Pressure (VP) values are computed for each PRM in the perfume mixture being tested. The VP of an individual PRM is calculated using the VP Computational Model, version 14.02 (Linux) available from Advanced Chemistry Development Inc. (ACD/Labs) (Toronto, Canada) to provide the VP value at 25° C. expressed in units of torr. The ACD/Labs' Vapor Pressure model is part of the ACD/Labs model suite.

The value of the log of the Octanol/Water Partition Coefficient (logP) for the perfume is computed for each PRM in the perfume mixture being tested. The logP of an individual PRM is calculated using the Consensus logP Computational Model, version 14.02 (Linux) available from Advanced Chemistry Development Inc. (ACD/Labs) (Toronto, Canada) to provide the unitless logP value. The ACD/Labs' Consensus logP Computational Model is part of the ACD/Labs model suite.

If particles are employed and PEG is the carrier, in addition to PEG, the particles can further comprise 0.1% to about 20% by weight perfume. The perfume can be unencapsulated perfume, encapsulated perfume, perfume provided by a perfume delivery technology, or a perfume provided in some other manner. The particles can comprise unencapsulated perfume and are essentially free of perfume carriers, such as a perfume microcapsules. The particles can comprise perfume carrier materials (and perfume contained therein).

The particles can comprise about 0.1% to about 20%, alternatively about 1% to about 15%, alternatively 2% to about 10%, alternatively combinations thereof and any whole percentages within any of the aforementioned ranges, of perfume by weight of the particles. The particles can comprise from about 0.1% by weight to about 6% by weight of the particles of perfume. The perfume can be unencapsulated perfume and or encapsulated perfume.

The particles can be free or substantially free of a perfume carrier. The particles may comprise about 0.1% to about 20%, alternatively about 1% to about 15%, alternatively 2% to about 10%, alternatively combinations thereof and any whole percentages within any of the aforementioned ranges, of unencapsulated perfume by weight of the particles.

The particles can comprise unencapsulated perfume and perfume microcapsules. Such levels of unencapsulated perfume can be appropriate for any of the particles disclosed herein that have unencapsulated perfume.

The particles can comprise unencapsulated perfume and perfume microcapsule but be free or essentially free of other perfume carriers. The particles, can comprise unencapsulated perfume and perfume microcapsules and be free of other perfume carriers.

The particles can comprise encapsulated perfume. Encapsulated perfume can be provided as plurality of perfume microcapsules. A perfume microcapsule is perfume oil enclosed within a shell. The shell can have an average shell thickness less than the maximum dimension of the perfume core. The perfume microcapsules can be friable perfume microcapsules. The perfume microcapsules can be moisture activated perfume microcapsules.

The perfume microcapsules can comprise a melamine/formaldehyde shell. Perfume microcapsules may be obtained from Appleton, Quest International, or International Flavor & Fragrances, or other suitable source. The perfume microcapsule shell can be coated with polymer to enhance the ability of the perfume microcapsule to adhere to fabric. This can be desirable if the particles are designed to be a fabric treatment composition. The perfume microcapsules can be those described in U.S. Patent Pub. 2008/0305982.

The particles can comprise about 0.1% to about 20%, alternatively about 1% to about 15%, alternatively about 2% to about 10%, alternatively about 0.1% to about 10%, alternatively about 0.4% to about 10%, alternatively combinations thereof and any whole percentages within any of the aforementioned ranges, of encapsulated perfume by weight of the particles.

The particles can comprise perfume microcapsules but be free of or essentially free of unencapsulated perfume. The particles may comprise about 0.1% to about 20%, alternatively about 1% to about 15%, alternatively about 0.1% to about 10%, alternatively about 0.4% to about 10%, alternatively combinations thereof and any tenths of percentages within any of the aforementioned ranges, of encapsulated perfume by weight of the particles.

The solid-form composition comprises a malodor reduction material. The malodor reduction material may not unduly interfere with the scent of the freshening compositions and devices that comprise such technologies and the perfumed or unperfumed situs that is treated with such freshening compositions and devices.

The malodor reduction materials may be a selected from the group consisting of: odor neutralizing materials such as reactive aldehydes (as disclosed in U.S. 2005/0124512); odor blocking materials; odor masking materials; sensory modifying materials such as ionones (also disclosed in U.S. 2005/0124512)); and combinations thereof.

The malodor reduction materials may be selected from the group consisting of: 1,1,2,3,3-pentamethyl-1,2,3,5,6,7-hexahydro-4H-inden-4-one, (E)-3,7-dimethylocta-2,6-dien-1-yl palmitate, (3R,3aR,6S,7S,8aS)-6-methoxy-3,6,8,8-tetramethyloctahydro-1H-3a,7-methanoazulene, Flor acetate, 1-((2-(tert-butyl)cyclohexyl)oxy)butan-2-ol, 3-Methyl-5-phenylpentanol, 2-(8-isopropyl-6-methylbicyclo[2.2.2]oct-5-en-2-yl)-1,3-dioxolane, 2,2,6,6,7,8,8-heptamethyldecahydro-2H-indeno[4,5-b]furan, 3,3-dimethyl-2,3-dihydro-1H-Indene-5-yl propanal, 1,1-dimethyl-2,3-dihydro-1H-inden-5-yl)propanal, 2,4-dimethyl-4,4a,5,9b-tetrahydroindeno[1,2-d][1,3]dioxine, (E)-oxacyclohexadec-13-en-2-one, (R,Z)-1-(2,6,6-trimethylcyclohex-2-en-1-yl)pent-1-en-3-one, 3-(7,7-dimethyl-4-bicyclo[3.1.1]hept-3-enyl)-2,2-dimethyl propanal, (Z)-cyclooct-4-en-1-yl methyl carbonate, octahydro-2H-chromen-2-one, 7-methyloctyl acetate, ethyl (2,3,6-trimethylcyclohexyl) carbonate, ethyl dodecanoate, (1R,4R,6R,10S)-4,12,12-trimethyl-9-methylene-5-oxatricyclo[8.2.0.04,6]dodecane, 4,8-dimethyl-1-(methylethyl)-7-oxybiciclo[4.3.0]nonane, 3a,4,5,6,7,7a-hexahydro-1H-4,7-methanoinden-6-yl propionate, 1-(5,5-dimethylcyclohex-1-en-1-yl)pent-4-en-1-one, 5-Cyclohexadecen-1-one, (E)-dec-4-enal, (1-methyl-2-((1,2,2-trimethylbicyclo[3.1.0]hexan-3-yl)methyl)cyclopropyl)methanol, 4-(tert-butyl)cyclohexan-1-ol, methyl palmitate, 2-isopropyl-5-methylphenol, 2,6-di-tert-butyl-4-methylphenol, and combinations thereof.

The solid-form composition may comprise from about 0.005% to about 10%, more preferably from about 0.025% to about 1%, most preferably from about 0.05% to about 0.5% of said malodor agent 0.005% to about 10%, more preferably from about 0.025% to about 1%, most preferably from about 0.05% to about 0.5% of said malodor reduction materials, by weight of the solid-form composition.

It is contemplated that the fluid composition may comprise other volatile materials in addition to or in substitution for the perfume mixture including, but not limited to, volatile dyes; compositions that function as insecticides or insect repellants; essential oils or materials that acts to condition, modify, or otherwise modify the environment (e.g. to assist with sleep, wake, respiratory health, and like conditions); deoderants; and combinations thereof.

Particles

The solid-form composition may be in the form of particles. If the solid-form composition is in the form of particles, the particles may have a variety of shapes. The particles may be formed into different shapes include tablets, pills, spheres, and the like. A particle can have a shape selected from the group consisting of spherical, hemispherical, compressed hemispherical, lentil shaped, and oblong. Lentil shaped refers to the shape of a lentil bean. Compressed hemispherical refers to a shape corresponding to a hemisphere that is at least partially flattened such that the curvature of the curved surface is less, on average, than the curvature of a hemisphere having the same radius. A compressed hemispherical particle can have a ratio of height to maximum based dimension of from about 0.01 to about 0.4, alternatively from about 0.1 to about 0.4, alternatively from about 0.2 to about 0.3. Oblong shaped refers to a shape having a maximum dimension and a maximum secondary dimension orthogonal to the maximum dimension, wherein the ratio of maximum dimension to the maximum secondary dimension is greater than about 1.2. An oblong shape can have a ratio of maximum base dimension to maximum minor base dimension greater than about 1.5. An oblong shape can have a ratio of maximum base dimension to maximum minor base dimension greater than about 2. Oblong shaped particles can have a maximum base dimension from about 2 mm to about 6 mm, a maximum minor base dimension of from about 2 mm to about 6 mm.

Individual particles can have a mass from about 0.1 mg to about 5 g, alternatively from about 10 mg to about 1 g, alternatively from about 10 mg to about 500 mg, alternatively from about 10 mg to about 250 mg, alternatively from about 0.95 mg to about 125 mg, alternatively combinations thereof and any whole numbers or ranges of whole numbers of mg or grams within any of the aforementioned ranges. In a plurality of particles, individual particles can have a shape selected from the group consisting of spherical, hemispherical, compressed hemispherical, lentil shaped, and oblong.

An individual particle may have a volume from about 0.003 cm³ to about 0.15 cm³. The plurality of particles can be made up of particles having different size, shape, and/or mass.

Each of the particles 50 can have a density less than 1 g/cm³. Each of the particles 50 can have a density less than about 0.95 g/cm³. Since the density of a typical washing solution is about 1 g/cm³, it can be desirable to provide particles 50 that have a density less than 1 g/cm³ or even less than about 0.95 g/cm³. By having the density less than 1 g/cm³ or even less than about 0.95 g/cm³, it is thought that with the typical manufacturing variability for particle making processes, that nearly all of the particles 50 produced will have a density less than about 1 g/cm³. Having nearly all of the particles 50 have a density less than about 1 g/cm³ can be desirable for providing for particles 50 that float in a wash liquor. The perfume bloom and release of malodor reduction materials that can occur from a wash liquor may be greater for particles 50 that float as compared to particles 50 that sink.

The particles 50 can have a maximum dimension of less than about 20 mm. The particles 50 can have a maximum dimension of less than about 10 mm.

Each of the particles 50 can have a volume and the occlusions of gas within the particles 50 can comprise between about 0.5% to about 50% by volume of the particle, or even between about 1% to about 20% by volume of the particle, or even between about 2% to about 15% by volume of the particle, or event between about 4% to about 12% by volume of the particle. Without being bound by theory, it is thought that if the volume of the occlusions of gas is too great, the particles 50 may not be sufficiently strong to be packaged, shipped, stored, and used without breaking apart in an undesirable manner.

The occlusions of gas can have an effective diameter between about 1 micron to about 2000 microns, or even between about 5 microns to about 1000 microns, or even between about 5 microns to about 200 microns, or even between about 25 to about 50 microns. In general, it is thought that smaller occlusions of gas are more desirable than larger occlusions of gas. If the effective diameter of the occlusions of gas are too large, it is thought that the particles 50 might not be sufficiently strong to be to be packaged, shipped, stored, and used without breaking apart in an undesirable manner. The effective diameter is diameter of a sphere having the same volume as the occlusion of gas. The occlusions of gas can be spherical occlusions of gas.

A precursor material comprising the composition of the particles 50 can be prepared by providing molten carrier into a mixer, which can optionally be a batch mixer. The mixer can be heated so as to help prepare the precursor material at the desired temperature. Perfume can added to the molten carrier, optionally in the mixer. Dye, if present, can be added to the mixer. Other adjunct materials can be added to the precursor material if desired. The precursor material can optionally be prepared by in-line mixing or other known approaches for mixing materials.

Screen

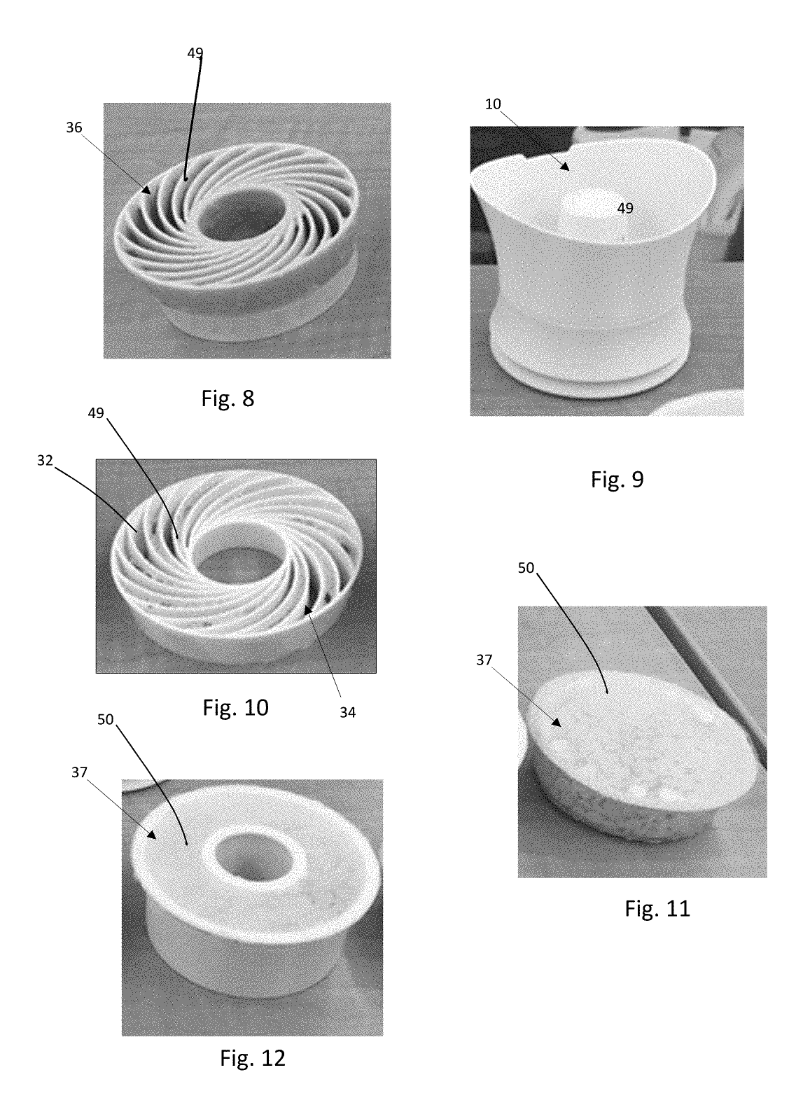

With reference to FIGS. 7-10, the air freshening device 10 may comprise a screen 32 disposed downstream of the fan. The screen 32 may be comprised of a rigid or semi-rigid material such as metal, plastic, resin, the like, and combinations thereof. The screen 32 may comprise a plurality of openings 34. The solid-form composition 49 may be disposed on at least a portion of the screen 32. With reference to FIG. 8, the solid-form composition 49 may completely surround the screen 32 or may partially surround the screen 32. The solid-form composition 49 may form a plurality of openings 36 surrounding the plurality of openings 34 in the screen 32. With reference to FIG. 10, the solid-form 49 composition may be disposed in a portion of the openings 34 of the screen 32.

### Pouch

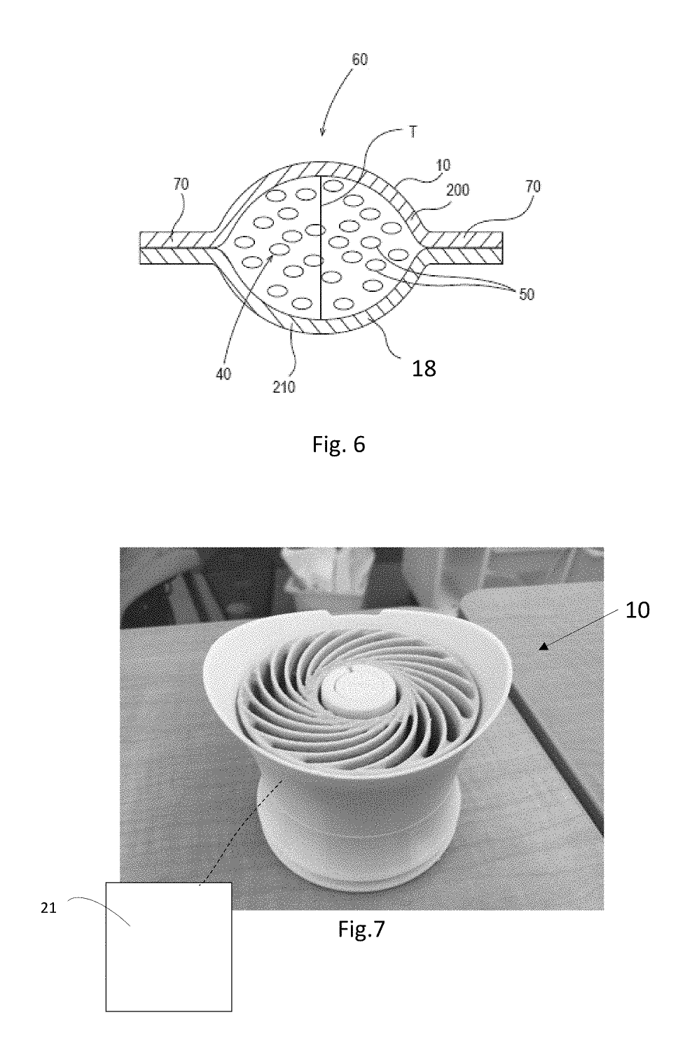

With reference to FIGS. 4-6, the substrate 18 can form at least part of a pouch 60 enclosing a chamber 40. The pouch 60 can comprise a bond 70 that at least partially defines, or defines the chamber 40. The bond 70 can be a thermal bond, fusion bond, adhesive bond, ultrasonic bond, or any other bond suitable for joining an edge of a material to another edge. The chamber 40 can contain a plurality of water soluble particles 50 that carry the perfume, as described herein. The particles 50 can be any of the particles 50 disclosed herein carrying perfume as described herein.

The pouch 60 can have a length L and width W each between about 2 cm to about 20 cm and a thickness of between about 5 mm and about 5 cm. The pouch 60 can have length L of about 8 to about 10 cm and a width W of about 3 cm to about 8 cm.

The pouch 60 can have an aperture sized and dimensioned to fit onto the hook 100 of a hanger.

The pouch 60 can have a tab 80 extending from the pouch 60. The tab 80 can be the bond 70. The tab 80 can be an extension from the bond 70. The tab 80 can be position so that the bond 70 or a portion of bond 70 is between an aperture and the chamber 40. The tab 80 can have an aperture 90 passing through the tab 80. The aperture can be sized and dimensioned to fit onto the hook 100 of a hanger. The aperture can have an open area between about 2 mm² and about 10 cm². The aperture 90 can be of any desired shape. The aperture 90 can be a slot.

A construction in which the pouch 60 has an aperture can be practical for associating a scent with a space. For instance, a consumer might hang the pouch 60 on the hook or hanger. A construction in which the pouch 60 has an aperture 90 can be appealing to consumers since the pouch 60 has multiple purposes, multiple uses, and can be used at multiple junctures in time to refresh the air. The consumer has the freedom and flexibility to choose to use the pouch 60 in a manner most congruent to his or her needs.

The chamber 40 can contain a plurality of particles 50. The plurality of particles 50 can have a mass between about 1 g and about 50 g of particles 50. The plurality of particles 50 can have a mass between about 1 g and about 100 g of particles 50. A cross section of a pouch 60 is shown in FIG. 4. As shown in FIG. 4, the pouch 60 can have a thickness T. The thickness T of the pouch can be between about 0.1 mm to about 20 mm. The thickness T of the pouch can be from about 3 mm to about 10 mm.

The fluid pervious pouch can comprise a substrate 18 having an apparent opening size according to ASTM D4751-12 smaller than about 1000 μm. The fluid pervious pouch can comprise a substrate 18 having an apparent opening size according to ASTM D4751-12 smaller than about 500 μm. The fluid pervious pouch can comprise a substrate 18 having an apparent opening size according to ASTM D4751-12 smaller than about 200 μm. The fluid pervious pouch can comprise a substrate 18 having an apparent opening size according to ASTM D4751-12 smaller than about 100 μm. The fluid pervious pouch can comprise a substrate 18 having an apparent opening size according to ASTM D4751-12 between about 50 μm and about 1000 μm. The fluid pervious pouch can comprise a substrate 18 having an apparent opening size according to ASTM D4751-12 between about 200 μm and about 800 μm. The fluid pervious pouch can comprise a substrate 18 having an apparent opening size according to ASTM D4751-12 between about 400 μm and about 600 μm.

This upper bound of apparent opening size can be practical to reduce the potential for the consumer's hands to contact the particles 50 when she employs the pouch 60 in the processes described herein. Depending on the constituents of the particles 50, the particles 50 may have a feel, texture, or chemical makeup that is not pleasing for the consumer to touch with her hand. Without being bound by theory, it is thought that by providing a substrate 18 having the aforesaid apparent opening size, the openings of such substrate 18 are small enough so as to reduce contact by the user with the particles 50 that are contained in the pouch 10 to a degree acceptable to the consumer. If larger openings are used, for instance an apparent opening size of 1 mm or greater, portions of the particles 50 may protrude through the openings of the substrate 18. This can be especially true if the particles 50 have a jagged shaped, irregularly shaped, or have sharply curved surfaces.

The pouch 60 can comprise a substrate 18 having a basis weight between about 1 gsm to about 100 gsm. In general, higher basis weight materials tend to have a greater thickness than lower basis weight materials, things such as material structure and constitution being equal. Higher basis weight substrates 10 can also be practical for reducing the potential for the consumer's hands to contact the particles 50. Without being bound by theory, this might be the case since a thicker substrate 18 increases the distance between the consumer's hand and the particles 50 contained in the pouch 60.

The substrate 18 can be air permeable. The substrate 18 can have an apparent opening size according to ASTM D4751-12 smaller than about 1000 μm and a basis weight between about 1 gsm to about 1000 gsm. The fluid pervious pouch can comprise a substrate 18 having an apparent opening size according to ASTM D4751-12 from about 50 μm to about 1000 μm. These ranges in these material properties are thought to provide for small enough openings and separation between the outside of the pouch 60 and the particles 50 contained therein to reduce to an acceptable level the potential for the consumer to contact the particles 50 with her hand.

A scented article can be used in the process as described herein. The scented article can comprise a fluid pervious pouch 60 enclosing a chamber 40 and a plurality of water soluble particles 50 contained in the chamber. The pouch 60 can comprise a substrate 18 having an apparent opening size according to ASTM D4751-12 smaller than about 1000 μm. The pouch 60 can comprise a substrate 18 having an apparent opening size according to ASTM D4751-12 from about 50 μm to about 1000 μm. The particles can comprise a carrier and a perfume. The particles 50 can have an individual mass between about 0.1 mg and about 10 g. The plurality of particles 50 can have a mass between about 5 g and about 100 g.

The pouch 60 can be used to associate a scent with a space as described herein. The pouch 60 may be place into the air freshening device 10 to provide perfume and malodor reduction materials into the air to freshen the air. It has been found that the use of a fan delivers good room fill of the perfume and malodor reduction materials from the solid-form composition 49 in the pouch 60 into the air.

For instance the pouch 60 can be placed in in a room, in a drawer, in a bag, in a hamper, in a pocket of clothing, and in various other locations where it is desired to transfer perfume and malodor reduction materials into the air to freshen the air.

An air-permeable substrate 18 forming the pouch 60 is a porous substrate. An air-permeable substrate is pervious to the flow of air and/or water. An air-permeable substrate can have a cross plane saturated hydraulic conductivity greater than about 1×10 cm/s. An air-permeable substrate can have an apparent opening size according to ASTM D4751-12 greater than, optionally greater than or equal to 0.075 mm

The substrate 18 can be water insoluble. The substrate 18 can be a fibrous web. For instance the substrate can be a fibrous web of the type commonly employed in dryer sheet products, including the substrate presently marketed as a dryer sheet under the BOUNCE brand, by The Procter & Gamble Company, Cincinnati, Ohio. The substrate 18 can be a fibrous nonwoven web. Fibrous webs such as those used in dryer sheets are thought to be durable enough to pass through both the wash and the drying cycle without the web disintegrating into multiple pieces.

The substrate 18 can be a polyester nonwoven fabric prepared from a polyester fiber having a denier of from about 2 to about 6. The substrate 18 can have a basis weight between about 1 gsm to about 100 gsm (gsm means grams per square meter throughout this description). The substrate 18 can have a basis weight between about 10 gsm to about 50 gsm. The substrate 18 can have a thickness between about 0.01 mm and about 20 mm. The substrate 18 can have a thickness between about 0.05 mm and about 2 mm. The substrate 18 can be a spun bonded web. The substrate 18 can be a web having a structure of individual fibers or threads which are interlaid, but not in a repeating pattern as in a woven or knitted fabric. The substrate can be a nonwoven web that is hydroentangled, spun laced, or bonded carded. The substrate can comprise polymeric fibers. Fibers that are polyolefinic can be suitable. Polypropylene and polyethylene fibers can also be suitable as either mono-comoponent fibers or bicomponent fibers. Other polymers such as polyvinyl alcohol, polyethylene, polyethylene terephthalate, and nylon can be suitable.

The substrate 18 can be a material that is compliant and soft feeling. A suitable substrate 18 can be manufactured from a wide range of materials such as polymeric materials, formed thermoplastic films, apertured plastic films, porous films, aperture formed films, reticulated foams, woven and non-woven synthetic fibers (e.g., polyester or polypropylene fibers) or from a combination of natural and synthetic fibers.

Optionally, the substrate 18 can be formed into a pouch that at least partially encloses particles that carry perfume.

The substrate 18 can be a nonwoven material available from FITESA, Washougal, Wash., United States of America, style 083YLJO09P, item description W4566, basis weight 27.8125 gsm. The substrate 18 can have a basis weight between about 20 gsm and about 40 gsm. The substrate 18 can have a thickness between about 0.05 mm and about 2 mm.

The substrate 18 can carry perfume on, within, or at least partially enclosed by the substrate. For example, the substrate 18 can be at least partially coated with a solid-form composition carrying the perfume. The carrier can be hot melt material that comprises the perfume. Optionally, the substrate 18 can carry a solid-form composition that in turn carries perfume within the matrix and the matrix is positioned within the interstitial spaces between fibers of the substrate 18. The substrate 18 can carry a solid-form composition on an external surface of the substrate 18 and in the substrate 18 in the interstitial spaces between fibers comprising the substrate 18.

The substrate 18 can have an apparent opening size according to ASTM D4751-12 smaller than about 1000 μm. The substrate 18 can have an apparent opening size according to ASTM D4751-12 from about 50 μm to about 1000 μm. Such a substrate 18 can have a pleasant hand to the consumer. And, if the substrate 18 is used in an embodiment in which particles are employed, such substrate 18 can help reduce the potential that the consumer's hand will come into contact with the particles during use. The substrate 18 can have a basis weight between about 1 gsm to about 100 gsm. Having such a basis weight can also help reduce the potential for the consumer to have contact with particles, if employed, and can provide for enough material to carry the desired amount of perfume.

The substrate 18 can be water insoluble. A substrate 18 is considered water insoluble if such substrate 18 can rest completely submerged in static deionized water for 24 hours at 20 degrees Celsius without losing more than 50% of its mass.

With reference to FIGS. 11 and 12, instead of a pouch, the particles 50 or solid-form composition 49 may be enclosed in a container 37. The container 37 may be inserted into the air freshening device 10 in place of the pouch 60.

### Sleeve

The fluid pervious substrate 18 can be packaged within a hermetically sealed sleeve 20, as shown in FIG. 4. The process can further comprise the step opening the sleeve 20 and removing the substrate 18 from the sleeve 20. Then the substrate 18 can be placed in in the device 10. The sleeve 20 can be formed of a plastic film material. Packaging the substrate 18 in a hermetically sealed sleeve 20 can be practical for reducing the amount of perfume lost from the substrate 18 via evaporation, which thereby leave more perfume available for providing the scent to the laundry article. The sleeve 20 can have a line of weakness 30. The line of weakness 30 can be a preferential line of weakness that can be torn or ripped to open the sleeve 20 to permit the consumer to retrieve the substrate 18. The substrate 18 can be individually packaged within a hermetically sealed sleeve 20 or packaged with a plurality of substrates 10 within a single sleeve 20. The pouch comprising the air-permeable substrate 18 can packaged in a hermetically sealed sleeve 20 likewise. If a pouch comprises the fluid pervious substrate 18, the pouch can be packaged within a hermetically sealed sleeve 20 in the same manner

### Housing

With reference to FIGS. 1-3 and 7, the device 10 may comprise a housing 12 for housing the solid-form composition in the form of a pouch 60 or screen 32, the fan assembly 80, and/or the cover 17. The housing 20 may be formed from a single part or from multiple parts that are joined together to define at least one chamber 13. The chamber 13 is in air flow communication with the fan 14. Air flows from the fan assembly 14 downstream to the solid-form composition. “Downstream”, as used herein, means a position in an airflow path that is later in time from a referenced position, when measuring air flow through the air freshening device. The housing 12 comprises one or more vents 38 for facilitating the passage of input air and output air into and out of the housing.

### Fan

The fan 13 can comprise any suitable fan or components configured to produce and/or move a volume of air over the solid-form composition.

The fan 13 can be housed in a fan housing. The fan can be positioned up to about 18 inches from the solid-form composition. The fan 13 may comprises a rotatable hub, and at least two fan blades extending from the rotatable hub or otherwise attached to or formed with the rotatable hub, and a motor.

The diameter of the rotatable hub may be about 8 mm to about 20 mm. The drive shaft can be operably engaged with the rotatable hub such that rotation of the drive shaft by the motor rotates the rotatable hub and thereby rotates the at least two fan blades.

The motor can provide continuous or intermittent movement of the fan blades to provide a volume of air over the solid-form composition. The fan 13 may produce air speeds in the range of about 5 feet per minute to about 400 feet per minute or from about 50 feet per minute to 250 feet per minute. The motor 88 may be a Mabuchi RF-J20WA-5Z145 motor that rotates the drive shaft at about 6200 revolutions per minute when 0.7 VDC is supplied to the motor 88 from the power source and rotates the driveshaft at about 9400 revolutions per minute when 1.0 VDC is supplied to the motor 88 from the power source. In various embodiments, the flow rate of the volume of air generated by the motor can be in the range of about 1.0 to about 8.0 mL/sec at about 0.7 VDC to about 6.0 to about 16.0 mL/sec at 1.0 VDC, depending upon the cross sectional area of the inlet orifice and the outlet orifice. By supplying various voltage levels to the motor 88, the rotational speed of the drive shaft and the resultant flow rate of the volume of air can be varied. Any other suitable motor can also be used with the fan, such as a Sunon UB393-10 fan assembly, for example. Additionally, the controller can supply the motor with voltage using any suitable technique known to those of skill in the art. In various embodiments, a pulse width modulation technique can be used to provide voltage to the motor over a specified range, such as about 0.7 VDC to about 1.0 VDC, for example. Additional circuitry or components, such as an analog-to-digital converter, can be used to compensate for various factors, such as the power source voltage and the ambient temperature, for example. In order to isolate or limit vibration due to the rotation of the drive shaft and/or the rotatable hub, vibration suppression devices or techniques can be used, such as silicon or thermoplastic elastomeric fan supports.

The fan 13 can comprises a centrifugal (i.e., radial) fan. Each fan blade can comprise an air forcing surface that is positioned in a direction parallel to, or substantially parallel to, an axis of rotation of the rotatable hub. In one embodiment, an electrical current can be provided to the motor via electrically conductive leads or terminal (not illustrated) to rotate the rotatable hub. Such rotation can cause a volume of air to be drawn into the fan inlet and forced in a radial direction relative to the drive shaft. In other embodiments, the volume of air can be drawn from the atmosphere outside of the device 10 through any suitable vent or passageway on the housing, for example. The rotation of the at least two fan blades can force the volume of air out of the fan housing through the fan outlet and over the solid-form composition. In various embodiments, the at least two fan blades can be arcuate, straight, and/or can have curved, straight, and/or arcuate portions. Additionally, the at least two fan blades can have various cross-sectional shapes, such as an airfoil shape or a tapered shape, for example. As will be appreciated by those of skill in the art, after consideration of the present disclosure, a centrifugal fan can provide high efficiency with relatively small dimensions, and changes in pressure may have little influence on pressure head drops through the device 10.

In another embodiment, the fan 13 can be an axial fan. This axial fan can comprise a rotatable hub and at least three fan blades extending from the rotatable hub. This at least three fan blades are attached to or formed with the rotatable hub. In one embodiment, the diameter of the rotatable hub can be about 8 mm to about 20 mm, for example, although others dimensions could be possible. The fan can define a fan inlet. The drive shaft can be operably engaged with the rotatable hub such that rotation of the drive shaft by the motor rotates the rotatable hub and thereby rotates the at least three fan blades. In various embodiments, the fan may produce air speeds in the range of about 5 feet per minute to 400 feet per minute or alternatively from about 50 feet per minute to 250 feet per minute; although others air speeds could be possible.

Each blade can comprise an air forcing surface that is positioned in a direction perpendicular to, or substantially perpendicular to, an axis of rotation of the rotatable hub. In one embodiment, an electrical current can be provided to the axial motor via electrically conductive leads or terminal (not illustrated) to rotate the rotatable hub. Such rotation can cause a volume of air to be drawn into the fan housing through the fan inlet. With an axial fan configuration, the air flowing through the fan assembly 80 can be drawn through the fan inlet and forced to move along the drive shaft direction. The rotation of the at least three fan blades can force the volume of air out of the fan housing through the fan outlet and over the microporous membrane. In various embodiments, the at least three fan blades 86 can be arcuate, straight, and/or can have curved, straight, and/or arcuate portions. Additionally, the at least three fan blades can have various cross-sectional shapes, such as an airfoil shape or a tapered shape, for example. As will be appreciated by those of skill in the art, after consideration of the present disclosure, an axial fan can provide high efficiency with relatively small dimensions, and changes in pressure may have little influence on pressure head drops through the solid form composition.

Suitable fans for the present invention include a 30×30×6 mm MagLev Motor Fan (Model MC30060V1-000U-A99), supplied by Sunon Wealth Electric Machine Industry Co., Ltd of Taiwan; and fan model RF-330TK 07800, supplied by Mabuchi Motor. Another suitable fan for the present invention may have the following specifications:

Dimension: 120×120×25 mm

Fan Speed: 800˜1500 rpm±250 RPM

Max Airflow: 66.55 CFM

Max Air Pressure: 1.42 mm H20

Bearing Type: Sleeve

Power: 5V

The fan is powered by a power source which may comprise a AC/DC outlet, a battery, such as a AA battery, a AAA battery, a 9-volt battery, rechargeable battery, and/or other suitable battery. In one embodiment, a solar power source, such as a solar cell, for example, can be used to power the device. In various embodiments, the solar cell (i.e., a photovoltaic cell) can be positioned on an outer portion of the device 10 or in communication with the device 10, such that the solar cell can receive light that can be transformed into energy to power the device 10. Those of skill in the art, upon review of the present disclosure, will recognize that any other suitable method or device can be used to provide power to the device 10.

Through control of the fan, maximum vapor phase volatile composition release can be achieved with a minimum amount of fan running time. In various embodiments, the sequencing or pattern of activator actuation, or the flow rate of the volume of air produced by the fan, can be adjusted to allow full or near full saturation of the volatile composition within the space for maximizing the vapor phase volatile composition release. In one embodiment, the fan can be activated for about 1 to about 10 seconds and then deactivated for about 1 to about 10 seconds, for example.

In various embodiments, the duration of activation of the fan or the flow rate of the volume of air provided by the fan can be increased to provide a higher intensity of volatile composition expulsion from the device 10. The fan can operate continuously or have intermittent operation. The fan may toggle on and off for a duty cycle of about 5% to about 50%, or from about 8% to about 20%. By providing a period of time between consecutive activations of the fan, a user is more likely to notice a scent of the volatile composition again and avoid habituation.

In various embodiments, a controller may be positioned in electrical communication with the fan, such that the controller can instruct the fan when to activate and which speed to rotate to force the volume of air over the microporous membrane 60. In one embodiment, the controller 95 can be any suitable type of controller, such as a microcontroller, for example. In one embodiment, the controller can be a Texas Instruments MSP430F2132 controller. In various embodiments, the controller can comprise one or more user input buttons or switches configured to provide an input signal to the controller when depressed by a user, such that the controller can send corresponding output signals to the fan and/or the user feedback module, for example.

In one embodiment, the various user input buttons or switches can comprise a power on/off switch 19 configured to power on or power off the device 10. As will be appreciated, the input buttons or switches can be any combination of buttons and/or switches, such as push buttons, sliders, dials, knobs, for example.

In some embodiments, a communication network may be implemented to gather information about the device for the user. For example, the device may be configured with a central device controller to form ad hoc, wireless mesh networks and control multiple communication modules. For example, the central device controller may be in communication with sensors to sense the amount of a volatile composition that has evaporated into a room having the device. Other types of sensors may exist on the device 10.

In various embodiments, the amount of the liquid volatile composition dispensed over a predetermined time interval can be controlled by adjusting the rate at which the fan is activated by the controller (i.e., by adjusting the time period the fan is active and the time period the fan is inactive), by adjusting the speed at which the air is moved when the fan is active (i.e., by adjusting the rotational speed by adjusting the voltage to the motor), and/or by a combination of both techniques. In one embodiment, the device 10 can have a “boost” button for delivering a dose of the volatile composition to the atmosphere on demand. For example, if the boost button is depressed or otherwise activated, the fan can be activated for a specified time period, such as 30 to 60 seconds or at a specified rotational speed, for example.

In various embodiments, the controller can also be in electrical communication with a temperature sensor configured to sense the temperature of the atmosphere. In various embodiments, the temperature sensor can send a signal to the controller indicative of the temperature of the space, such that the controller can provide an output signal to the fan or other various components of the device 10, indicative of a volatile composition dosing amount for a particular temperature and/or temperature range. For example, higher temperature ranges may require greater dose amounts than lower temperature ranges to achieve the desired result. As a result, the device 10 can be power efficient such that it can maximize the life of the power source 100. The device 10 can be activated for 1-30 seconds, for example, and then be inactive for 10-200 seconds, for example. In other various embodiments, the device 10 can be set by a user to provide a desired intermittent dosing amount.

In various embodiments, the device 10 can comprise a sensor, such as a visible indicator, a light source, and/or an audible alert, configured to provide feedback to the user regarding the status of the device 10. In one embodiment, the sensor can be used to alert the user of a property of the device 10. In such embodiments, the feedback can be visual and/or audible and can indicate to the user, among other things, whether the device 10 is powered on, what volatile composition dosing amount is being dispensed, the power level of the power source, the amount, type, or level of the solid-form composition within the pouch 60 or screen 32, and/or any other suitable feedback helpful or beneficial to the user. In various embodiments, the sensor can comprise one or more one indicators, such as a plurality of light sources, for example, electrically coupled to the controller and/or to the power source, and a translucent portion in the housing, such that the one or more indicators can be viewed by the user though the housing. In one embodiment, the one or more indicators can be oriented in any suitable fashion such that various lights of the one or more indicators can emit visible light through the translucent portion of the housing 12, depending on what type of feedback is being provided to the user. In one embodiment, the translucent portion of the housing 12 can comprise any suitable shape and the one or more indicators can be arranged in a similar shape so that as one indicator, such as a light source, for example, is powered or unpowered, the user is provided with a first feedback and, as two or more light sources are powered or unpowered, the user is provided with at least a second feedback and so forth. In one embodiment, at least one button is at least partially translucent allowing for one or more indicators to be viewable through the button.

With some volatile compositions (for instance those comprising fragrances) it may be helpful to adjust the fan speed, frequency of run time, or on/off time to compensate for the changing volatile composition formulation as high vapor pressure volatile composition raw materials will evaporate more quickly than low vapor pressure raw materials. In this case it may optionally be desirable to have the controller operate the fan more frequently as the volatile composition is evaporated over a period of many days. For instance in one non-limiting example, the fan could run at 10% duty cycle for the first 10 days of usage but then slowly increase up to about 30% to about 40% duty cycle from days 11 up to 60 days. In this way, the fan frequency or duration can be increased to compensate for potentially a decline in fragrance intensity. By adjusting for the age, it is possible to deliver a more consistent scent intensity even as the fragrance amount and mixture of high to low vapor pressure components is changing with time. One non-limiting example of a means of keeping track of run time of the solid-form composition is to monitor the voltage of the battery associated with the delivery engine 50. For instance, a new AA battery may be 1.60 Volts to about 1.65 Volts while a AA battery that was used for thirty days might have a voltage of about 1.2 Volts to about 1.45 Volts. By monitoring the voltage of the battery, the controller can recognize the life of the solid-form composition and can adjust operating conditions to deliver a consistent scent experience over the life of the solid-form composition.

Another non-limiting example of a means to monitor time, is to start a timer when the solid-form composition in the form of a pouch or screen is inserted and to keep track of hours/minutes that the fan has operated. As mentioned above, the fan time could be adjusted as the product ages to deliver a more consistent scent experience.

In the instance where the battery voltage or run time is viewed as the indicator of the full life of the solid-form composition, the controller could be programmed to provide a signal to the user such as turning on a red light or provide a flashing light to indicate that the solid-form composition needs to be replaced.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An air freshening device comprising:
a housing;
a fan connected with the housing; and
a pouch disposed in air-flow communication with the fan, wherein the pouch is disposed downstream from the fan, wherein the pouch comprises a solid-form composition enclosed by an air-permeable substrate, the solid form composition comprising a perfume mixture and a malodor reduction material.

2. The air freshening device of claim 1, wherein the solid-form composition is in the form of particles.

3. The air freshening device of claim 1, wherein the malodor reduction material comprises a material selected from the group consisting of: 1,1,2,3,3-pentamethyl-1,2,3,5,6,7-hexahydro-4H-inden-4-one, (E)-3,7-dimethylocta-2,6-dien-1-yl palmitate, (3R,3aR,6S,7S ,8aS)-6-methoxy-3,6,8,8-tetramethyloctahydro-1H-3a,7-methanoazulene, Flor acetate, 1-((2-(tert-butyl)cyclohexyl)oxy)butan-2-ol, 3-Methyl-5-phenylpentanol, 2-(8-isopropyl-6-methylbicyclo[2.2.2]oct-5-en-2-yl)-1,3-dioxolane, 2,2,6,6,7,8,8-heptamethyldecahydro-2H-indeno[4,5-b]furan, 3,3-dimethyl-2,3-dihydro-1H-Indene-5-yl propanal, 1,1-dimethyl-2,3-dihydro-1H-inden-5-yl)propanal, 2,4-dimethyl-4,4a,5,9b-tetrahydroindeno[1,2-d][1,3]dioxine, (E)-oxacyclohexadec-13-en-2-one, (R,Z)-1-(2,6,6-trimethylcyclohex-2-en-1-yl)pent-1-en-3-one, 3-(7,7-dimethyl-4-bicyclo[3.1.1]hept-3-enyl)-2,2-dimethyl propanal, (Z)-cyclooct-4-en-1-yl methyl carbonate, octahydro-2H-chromen-2-one, 7-methyloctyl acetate, ethyl (2,3,6-trimethylcyclohexyl) carbonate, ethyl dodecanoate, (1R,4R,6R,10S)-4,12,12-trimethyl-9-methylene-5-oxatricyclo[8.2.0.04,6]dodecane, 4,8-dimethyl-1-(methylethyl)-7-oxybiciclo [4.3.0]nonane, 3a,4,5,6,7,7a-hexahydro-1H-4,7-methanoinden-6-yl propionate, 1-(5,5-dimethylcyclohex-1-en-1-yl)pent-4-en-1-one, 5-Cyclohexadecen-1-onc, (E)-dec-4-enal, (1-methyl-2-((1,2,2-trimethylbicyclo[3.1.0]hexan-3-yl)methyl)cyclopropyl)methanol, 4-(tert-butyl)cyclohexan-1-ol, methyl palmitate, 2-isopropyl-5-methylphenol, 2,6-di-tert-butyl-4-methylphenol, and combinations thereof.

4. The air freshening device of claim 1, wherein the malodor reduction material comprises a material selected from the group consisting of: odor neutralizing materials, odor blocking materials, odor masking materials, sensory modifying materials, and combinations thereof.

5. The air freshening device of claim 1 further comprising a cover, wherein the screen encloses the pouch in the housing.

6. The air freshening device of claim 1, wherein the solid-form composition comprises about 0.1% to about 20%, by weight of the solid form composition, of perfume.

7. The air freshening device of claim 1, wherein the solid-form composition comprises about 0.005% to about 10%, by weight of the solid-form composition, of malodor reduction material.

8. The air freshening device of claim 1, wherein the air-permeable substrate comprises a fibrous web having an apparent opening size according to ASTM D4751-12 smaller than about 1000 μm.

9. An air freshening device comprising:
a housing;
a fan connected with the housing; and
a screen in air flow communication with the fan, wherein the screen comprises a plurality of openings and a semi-solid or solid form composition disposed on at least a portion of the screen, wherein the solid-form composition comprises a perfume mixture and a malodor reduction material.

10. The air freshening device of claim 9, wherein the solid-form composition completely surrounds the screen.

11. The air freshening device of claim 9, wherein the solid-form composition comprises a carrier.

12. The air freshening device of claim 9, wherein the solid-form composition forms a plurality of openings around the plurality of openings of the screen.

13. The air freshening device of claim 9, wherein the malodor reduction material comprises a material selected from the group consisting of: 1,1,2,3,3-pentamethyl-1,2,3,5,6,7-hexahydro-4H-inden-4-one, (E)-3,7-dimethylocta-2,6-dien-1-yl palmitate, (3R,3aR,6S,7S,8aS)-6-methoxy-3,6,8,8-tetramethyloctahydro-1H-3a,7-methanoazulene, for acetate, 1-((2-(tert-butyl)cyclohexyl)oxy)butan-2-ol, 3-Methyl-5-phenylpentanol, 2-(8-isopropyl-6-methylbicyclo[2.2.2]oct-5-en-2-yl)-1,3-dioxolane, 2,2,6,6,7,8,8-heptamethyldecahydro-2H-indeno[4,5-b]furan, 3,3-dimethyl-2,3-dihydro-1H-Indene-5-yl propanal, 1,1-dimethyl-2,3-dihydro-1H-inden-5-yl)propanal, 2,4-dimethyl-4,4a,5,9b-tetrahydroindeno[1,2-d][1,3]dioxine, (E)-oxacyclohexadec-13-en-2-one, (R,Z)-1-(2,6,6-trimethylcyclohex-2-en-1-yl)pent-1-en-3-one, 3-(7,7-dimethyl-4-bicyclo[3.1.1]hept-3-enyl)-2,2-dimethyl propanal, (Z)-cyclooct-4-en-1-yl methyl carbonate, octahydro-2H-chromen-2-one, 7-methyloctyl acetate, ethyl (2,3,6-trimethylcyclohexyl) carbonate, ethyl dodecanoate, (1R,4R,6R,10S)-4,12,12-trimethyl-9-methylene-5-oxatricyclo[8.2.0.04,6]dodecane, 4,8-dimethyl-1-(methylethyl)-7-oxybiciclo [4.3.0]nonane, 3a,4,5,6,7,7a-hexahydro-1H-4,7-methanoinden-6-yl propionate, 1-(5,5-dimethylcyclohex-1-en-1-yl)pent-4-en-1-one, 5-Cyclohexadecen-1-one, (E)-dec-4-enal, (1-methyl-2-((1,2,2-trimethylbicyclo[3.1.0]hexan-3-yl)methyl)cyclopropyl)methanol, 4-(tert-butyl)cyclohexan-1-ol, methyl palmitate, 2-isopropyl-5-methylphenol, 2,6-di-tert-butyl-4-methylphenol, and combinations thereof.

14. The air freshening device of claim 9, wherein the malodor reduction material comprises a material selected from the group consisting of: odor neutralizing materials, odor blocking materials, odor masking materials, sensory modifying materials, and combinations thereof.

15. The air freshening device of claim 9 further comprising a cover, wherein the cover encloses the screen in the housing.

16. The air freshening device of claim 9, wherein the solid-form composition comprises about 0.1% to about 20%, by weight of the solid form composition, of perfume.

17. The air freshening device of claim 9, wherein the solid-form composition comprises about 0.005% to about 10%, by weight of the solid-form composition, of malodor reduction material.

18. A method of freshening the air with the air freshening device of claim 1, wherein the method includes the step of:
providing the air freshening device;
inserting a pouch into the housing; and
turning on power to fan.

19. A method of freshening the air with the air freshening device of claim 9, wherein the method includes the step of:
providing the air freshening device;
inserting a screen into the housing; and
turning on power to fan.

20. The method of claim 18 further comprising the step of replacing the pouch with a new pouch.
